(19) United States
(12) Patent Application Publication
(10) Pub. No.: US 2016/0030643 A1
(43) Pub. Date: Feb. 4, 2016

(54) DRUG COATED STENTS

(71) Applicant: Micell Technologies, Inc., Durham, NC (US)

(72) Inventor: James B. McClain, Ocracoke, NC (US)

(73) Assignee: MICELL TECHNOLOGIES, INC., Durham, NC (US)

(21) Appl. No.: 14/718,467

(22) Filed: May 21, 2015

Related U.S. Application Data
(63) Continuation of application No. 12/738,411, filed on Jun. 9, 2010, now abandoned.
(60) Provisional application No. 61/104,669, filed on Oct. 10, 2008.
(60) Provisional application No. 61/045,928, filed on Apr. 17, 2008.
(60) Provisional application No. 60/981,445, filed on Oct. 19, 2007.

(51) Int. Cl. A61L 31/16; A61L 31/02; A61L 31/04; A61L 31/10

(52) U.S. Cl. CPC A61L 31/16; A61L 31/10; A61L 31/022; A61L 31/042; A61L 2420/06; A61L 2420/08; A61L 2300/426; A61L 2300/42; A61L 2420/02; A61L 2300/236

(57) ABSTRACT

Provided herein is a coated coronary stent including a stent framework, heparin molecules attached to the stent framework and a rapamycin-polymer coating wherein at least part of rapamycin is a crystalline form. In one embodiment, the rapamycin-polymer coating comprises one or more resorbable polymers. Methods for preparing stents are also disclosed.

### CROSS-REFERENCE TO RELATED APPLICATIONS

This application is a continuation of U.S. application Ser. No. 12/738,411 filed Jun. 9, 2010, which claims the benefit PCT/US08/11852 filed Oct. 17, 2008, which claims benefit of U.S. Provisional Application No. 61/104,669 filed Oct. 10, 2008, which claims benefit of U.S. Provisional Application No. 61/045,928 filed Apr. 17, 2008, which claims benefit of U.S. Provisional Application No. 60/981,445 filed Oct. 19, 2007, the disclosures of which are incorporated herein by reference.

### BACKGROUND OF THE INVENTION

The present invention relates to methods for depositing a coating comprising a polymer and a pharmaceutical or biological agent in powder form onto a substrate.

It is often beneficial to provide coatings onto substrates, such that the surfaces of such substrates have desired properties or effects.

For example, it is useful to coat biomedical implants to provide for the localized delivery of pharmaceutical or biological agents to target specific locations within the body, for therapeutic or prophylactic benefit. One area of particular interest is that of drug eluting stents (DES) that has recently been reviewed by Ong and Serruys in Nat. Clin. Pract. Cardiovasc. Med., (December 2005), Vol 2, No 12, 647. Typically such pharmaceutical or biological agents are co-deposited with a polymer. Such localized delivery of these agents avoids the problems of systemic administration, which may be accompanied by unwanted effects on other parts of the body, or because administration to the afflicted body part requires a high concentration of pharmaceutical or biological agent that may not be achievable by systemic administration. The coating may provide for controlled release, including long-term or sustained release, of a pharmaceutical or biological agent. Additionally, biomedical implants may be coated with materials to provide beneficial surface properties, such as enhanced biocompatibility or lubriciousness.

Conventionally, coatings have been applied by processes such as dipping, spraying, vapor deposition, plasma polymerization, and electro-deposition. Although these processes have been used to produce satisfactory coatings, there are drawbacks associated therewith. For example it is often difficult to achieve coatings of uniform thicknesses and prevent the occurrence of defects (e.g. bare spots). Also, in many processes, multiple coating steps are frequently necessary, usually requiring drying between or after the coating steps.

Another disadvantage of most conventional methods is that many pharmaceutical or biological agents, once deposited onto a substrate, suffer from poor bioavailability, reduced shelf life, low in vivo stability or uncontrollable elution rates, often attributable to poor control of the morphology and/or secondary structure of the agent. Pharmaceutical agents present significant morphology control challenges using existing spray coating techniques, which conventionally involve a solution containing the pharmaceutical agents being sprayed onto a substrate. As the solvent evaporates the agents are typically left in an amorphous state. Lack of or low degree of crystallinity of the spray coated agent can lead to decreased shelf life and too rapid drug elution. Biological agents typically rely, at least in part, on their secondary, tertiary and/or quaternary structures for their activity. While the use of conventional solvent-based spray coating techniques may successfully result in the deposition of a biological agent upon a substrate, it will often result in the loss of at least some of the secondary, tertiary and/or quaternary structure of the agent and therefore a corresponding loss in activity. For example, many proteins lose activity when formulated in carrier matrices as a result of the processing methods.

Conventional solvent-based spray coating processes are also hampered by inefficiencies related to collection of the coating constituents onto the substrate and the consistency of the final coating. As the size of the substrate decreases, and as the mechanical complexity increases, it grows increasingly difficult to uniformly coat all surfaces of a substrate.

### BRIEF SUMMARY OF THE INVENTION

One embodiment provides a coated coronary stent, comprising: a stent framework; heparin molecules attached to the stent framework; and a rapamycin-polymer coating wherein at least part of rapamycin is in crystalline form. In one embodiment, the rapamycin-polymer coating comprises one or more resorbable polymers.

In another embodiment the rapamycin-polymer coating has substantially uniform thickness and rapamycin in the coating is substantially uniformly dispersed within the rapamycin-polymer coating.

In another embodiment, the one or more resorbable polymers are selected from PLGA (poly(lactide-co-glycolide); DPLA-poly(dl-lactide); LPLA-poly(l-lactide); PGA-polyglycolide; PDO-poly(dioxanone); PGA-TMC-poly(glycolide-co-trimethylene carbonate); PGA-LPLA-poly (l-lactide-co-glycolide); PGA-DLPLA-poly(dl-lactide-glycolide); LPLA-DLPLA-poly(l-lactide-co-dl-lactide); PDO-PGA-TMC-poly(glycolide-co-trimethylene carbonate-co-dioxanone) and combinations thereof.

In yet another embodiment the polymer is 50/50 PLGA.

In still another embodiment the at least part of said rapamycin forms a phase separate from one or more phases formed by said polymer.

In another embodiment the rapamycin is at least 50% crystalline.

In another embodiment the rapamycin is at least 75% crystalline.

In another embodiment the rapamycin is at least 90% crystalline.

In another embodiment the rapamycin is at least 95% crystalline.

In another embodiment the rapamycin is at least 99% crystalline.

In another embodiment the polymer is a mixture of two or more polymers.

In another embodiment the mixture of polymers forms a continuous film around particles of rapamycin.

In another embodiment the two or more polymers are intimately mixed.

In another embodiment the mixture comprises no single polymer domain larger than about 20 nm.

In another embodiment the each polymer in said mixture comprises a discrete phase.

In another embodiment the discrete phases formed by said polymers in said mixture are larger than about 10 nm.

In another embodiment the discrete phases formed by said polymers in said mixture are larger than about 50 nm.

In another embodiment the rapamycin in said stent has a shelf stability of at least 3 months.

In another embodiment the rapamycin in said stent has a shelf stability of at least 6 months.

In another embodiment the rapamycin in said stent has a shelf stability of at least 12 months.

In another embodiment the coating is substantially conformal.

In another embodiment the stent provides an elution profile wherein about 10% to about 50% of rapamycin is eluted at week 1 after the composite is implanted in a subject under physiological conditions, about 25% to about 75% of rapamycin is eluted at week 2 and about 50% to about 100% of rapamycin is eluted at week 6.

In another embodiment the onset of heparin anti-coagulant activity is obtained at week 3 or later.

In another embodiment heparin anti-coagulant activity remains at an effective level at least 90 days after onset of heparin activity.

In another embodiment heparin anti-coagulant activity remains at an effective level at least 120 days after onset of heparin activity.

In another embodiment heparin anti-coagulant activity remains at an effective level at least 200 days after onset of heparin activity.

In another embodiment the stent framework is a stainless steel framework.

In another embodiment heparin is attached to the stainless steel framework by reaction with an aminated silane.

In another embodiment the framework is coated with a silane monolayer.

A further embodiment provides a coated coronary stent, comprising: a stent framework; heparin molecules attached to the stent framework by an aminated silane; and a rapamycin-polymer coating wherein at least part of rapamycin is in crystalline form and wherein the polymer is bioabsorbable.

In Still another embodiment provides a coated coronary stent, comprising: a stent framework having a heparin coating disposed thereon; and a macrolide immunosuppressive (limus) drug-polymer coating wherein at least part of the drug is in crystalline form.

In another embodiment the macrolide immunosuppressive drug comprises one or more of rapamycin, 40-0-(2-Hydroxyethyl)rapamycin (everolimus), 40-0-Benzyl-rapamycin, 40-0-(4′-Hydroxymethyl)benzyl-rapamycin, 40-0-[4′-(1,2-Dihydroxyethye)]benzyl-rapamycin,40-0-Allyl-rapamycin, 40-0-Allyl-rapamycin, 40-0-[3′-(2,2-Dimethyl-1,3-dioxolan-4(S)-yl)-prop-2′-en-1′-yl]-rapamycin, (2′:E,4′S)-40-0-(4′,5,40-Dihydro xypent-2′-en-1′-yl)-rapamycin40-0-(2-Hydroxy) ethoxycar-bonylmethyl-rapamycin, 40-0-(3-Hydroxy)propyl-rapamycin 40-0-(6-Hydroxy)hexyl-rapamycin 40-0-[2-(2-Hydroxy)ethoxyl]ethyl-rapamycin 40-0-[(3S)-2,2-Dimethyldioxolan-3-yl]methyl-rapamycin, 40-0-[(2S)-2,3-Dihydroxyprop-1-yl]-rapamycin, 40-0-(2-Acetoxy)ethyl-rapamycin 40-0-(2-Nicotinoyloxy) ethyl-rapamycin, 40-0-[2-(N-Morpholino)acetoxyl]ethyl-rapamycin 40-0-(2-N-lmidazolylacetoxy)ethyl-rapamycin, 40-0-[2-(N-Methyl-N′-piperazinyl)acetoxy]ethyl-rapamycin, 39-0-Desmethyl-39,40-0,0-ethylene-rapamycin, (26R)-26-Dihydro-40-0-(2-hydroxy)ethyl-rapamycin, 28-0-Methyl-rapamycin, 40-0-(2-Aminoethyl)-rapamycin, 40-0-(2-Acetaminoethyl)-rapamycin 40-0-(2-Nicotinamidoethyl)-rapamycin, 40-0-(2-(N-Methyl-imidazo-2′-ylcarbethoxamido)ethyl)-rapamycin, 40-0-(2-Ethoxycarbonylaminoethyl)-rapamycin, 40-0-(2-Tolylsulfonamidoethyl)-rapamycin, 40-0-[2-(4′,5′-Dicarboethoxy-1′,2′,3′-triazol-1′yl)-ethyl]-rapamycin, 42-Epi-(tetrazolyl)rapamycin (tacrolimus), and 42-[3-hydroxy-2-(hydroxymethyl)-2-methylpropanoate]rapamycin (temsirolimus).

In another embodiment the macrolide immunosuppressive drug is at least 50% crystalline.

Another embodiment provides a method for preparing a coated coronary stent comprising the following steps: forming a silane layer on a stainless or cobalt-chromium stent framework; covalently attaching heparin molecules to the silane layer; forming a macrolide immunosuppressive (limus) drug-polymer coating on the stent framework wherein at least part of the drug is in crystalline form.

In another embodiment the macrolide is deposited in dry powder form.

In another embodiment the bioabsorbable polymer is deposited in dry powder form.

In another embodiment the polymer is deposited by an e-SEDS process.

In another embodiment the polymer is deposited by an e-RESS process.

Another embodiment provides a method further comprising sintering said coating under conditions that do not substantially modify the morphology of said macrolide.

Yet another embodiment provides a coated coronary stent, comprising: a stent framework; heparin molecules attached to the stent framework; a first layer of bioabsorbable polymer; and a rapamycin-polymer coating wherein comprising rapamycin and a second bioabsorbable polymer wherein at least part of rapamycin is in crystalline form and wherein the first polymer is a slow absorbing polymer and the second polymer is a fast absorbing polymer.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

Illustration of selected embodiments of the inventions is provided in appended FIGS. 1-5.

FIG. 1 is a cross sectional representation of a stent according one embodiment of the present invention.

FIG. 2 is a chart detailing the elution profile of a stent according to one embodiment of the present invention.

FIG. 3 illustrates a coating according to one embodiment of the present invention with and without polymer material.

FIG. 4 is an enlarged view of certain portion of a stent according to one embodiment of the present invention.

FIG. 5 illustrates a system utilized in creation of a stent according to one embodiment of the present invention.

### DETAILED DESCRIPTION

The present invention is explained in greater detail below. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure, which do not depart from the instant invention. Hence, the following specification is intended to illustrate some particular embodiments of the invention, and not to exhaustively specify all permutations, combinations and variations thereof.

One embodiment provides a coated coronary stent, comprising: a stent framework; heparin molecules attached to the stent framework; and a rapamycin-polymer coating wherein at least part of rapamycin is in crystalline form. In one embodiment, the rapamycin-polymer coating comprises one or more resorbable polymers.

### Definitions

As used in the present specification, the following words and phrases are generally intended to have the meanings as set forth below, except to the extent that the context in which they are used indicates otherwise.

Examples of therapeutic agents employed in conjunction with the invention include, rapamycin, 40-O-(2-Hydroxyethyl)rapamycin (everolimus), 40-O-Benzyl-rapamycin, 40-O-(4′-Hydroxymethyl)benzyl-rapamycin, 40-O-[4′-(1,2-Dihydroxyethyl)]benzyl-rapamycin, 40-0-Allyl-rapamycin, 40-O-[3′-(2,2-Dimethyl-1,3-dioxolan-4(S)-yl)-prop-2′-en-1′-yl]-rapamycin, (2′:E,4′S)-40-O-(4′,5′-Dihydroxypent-2′-en-1′-yl)-rapamycin 40-O-(2-Hydroxy)ethoxycar-bonylmethyl-rapamycin, 40-O-(3-Hydroxy)propyl-rapamycin 40-O-(6-Hydroxy)hexyl-rapamycin 40-O-[2-(2-Hydroxy)ethoxy)ethyl-rapamycin 40-O-(3S)-2,2-Dimethyldioxolan-3-yl]methyl-rapamycin, 40-O-[(2S)-2,3-Dihydroxyprop-1-yl]-rapamycin, 40-O-(2-Acetoxy)ethyl-rapamycin 40-O-(2-Nicotinoyloxy)ethyl-rapamycin, 40-O-[2-(N-Morpholino)acetoxy]ethyl-rapamycin 40-O-(2-N-Imidazolylacetoxy)ethyl-rapamycin, 40-O-[2-(N-Methyl-N′-piperazinl)acetoxy]ethyl-rapamycin, 39-0-Desmethyl-39,40-O,0-ethylene-rapamycin, (26R)-26-Dihydro-40-O-(2-hydroxy)ethyl-rapamycin, 28-0-Methyl-rapamycin, 40-O-(2-Aminoethyl)-rapamycin, 40-O-(2-Acetaminoethyl)-rapamycin 40-O-(2-Nicotinamidoethyl)-rapamycin, 40-O-(2-(N-Methyl-imidazo-2′-ylcarbethoxamido)ethyl)-rapamycin, 40-O-(2-Ethoxycarbonylaminoethyl)-rapamycin, 40-O-(2-Tolylsulfonamidoethyl)-rapamycin, 40-O-[2-(4′,5′-Dicarboethoxy-1′,2′,3′-triazol-1′-yl)-ethyl]-rapamycin, 42-Epi-(tetrazolyl)rapamycin (tacrolimus), and 42-[3-hydroxy-2-hydroxymethyl)-2-methylpropanoate]rapamycin (temsirolimus).

The active ingredients may, if desired, also be used in the form of their pharmaceutically acceptable salts or derivatives (meaning salts which retain the biological effectiveness and properties of the compounds of this invention and which are not biologically or otherwise undesirable), and in the case of chiral active ingredients it is possible to employ both optically active isomers and racemates or mixtures of diastereoisomers.

“Stability” as used herein in refers to the stability of the drug in a polymer coating deposited on a substrate in its final product form (e.g., stability of the drug in a coated stent). The term stability will define 5% or less degradation of the drug in the final product form.

“shelflife” is referred to herein mainly in connection with a product wherein the pharmaceutical agent or agents are stable as defined above for a desired period of time. To achieve the desired shelf life for the product as a whole other parameters which are outside the scope of this application should also be controlled (packaging, storage, etc.)

“Heparin activity” as referred to herein indicates that heparin molecules attached to the stent framework become exposed after bioabsorbable polymer that may be covering the molecules is absorbed thereby uncovering the heparin molecules and making them available for acting as anti-coagulant agents. This is to be contrasted with the situation where the heparin molecules are covered by a polymer layer and therefore cannot be accessed for anticoagulant activity. As more of the polymer layer is absorbed more heparin molecules are uncovered thereby increasing anticoagulant activity of the heparin coated stent framework.

“Secondary, tertiary and quaternary structure ” as used herein are defined as follows. The active biological agents of the present invention will typically possess some degree of secondary, tertiary and/or quaternary structure, upon which the activity of the agent depends. As an illustrative, non-limiting example, proteins possess secondary, tertiary and quaternary structure. Secondary structure refers to the spatial arrangement of amino acid residues that are near one another in the linear sequence. The a.-helix and the I)-strand are elements of secondary structure. Tertiary structure refers to the spatial arrangement of amino acid residues that are far apart in the linear sequence and to the pattern of disulfide bonds. Proteins containing more than one polypeptide chain exhibit an additional level of structural organization. Each polypeptide chain in such a protein is called a subunit. Quaternary structure refers to the spatial arrangement of subunits and the nature of their contacts. For example hemoglobin consists of two a and two J3 chains. It is well known that protein function arises from its conformation or three dimensional arrangement of atoms (a stretched out polypeptide chain is devoid of activity). Thus one aspect of the present invention is to manipulate active biological agents, while being careful to maintain their conformation, so as not to lose their therapeutic activity.

“Polymer” as used herein, refers to a series of repeating monomeric units that have been cross-linked or polymerized. Any suitable polymer can be used to carry out the present invention. It is possible that the polymers of the invention may also comprise two, three, four or more different polymers. In some embodiments, of the invention only one polymer is used. In some preferred embodiments a combination of two polymers are used. Combinations of polymers can be in varying ratios, to provide coatings with differing properties. Those of skill in the art of polymer chemistry will be familiar with the different properties of polymeric compounds.

“The rapeutically desirable morphology” as used herein refers to the gross form and structure of the pharmaceutical agent, once deposited on the substrate, so as to provide for optimal conditions of ex vivo storage, in vivo preservation and/or in vivo release. Such optimal conditions may include, but are not limited to increased shelf life, increased in vivo stability, good biocompatibility, good bioavailability or modified release rates. Typically, for the present invention, the desired morphology of a pharmaceutical agent would be crystalline or semi-crystalline or amorphous, although this may vary widely depending on many factors including, but not limited to, the nature of the pharmaceutical agent, the disease to be treated/prevented, the intended storage conditions for the substrate prior to use or the location within the body of any biomedical implant. Preferably at least 10%, 20%, 30%, 40%, 50%, 60%, 70%, 80%, 90% or 100% of the pharmaceutical agent is in crystalline or semi-crystalline form.

“Stabilizing agent” as used herein refers to any substance that maintains or enhances the stability of the biological agent. Ideally these stabilizing agents are classified as Generally Regarded As Safe (GRAS) materials by the US Food and Drug Administration (FDA). Examples of stabilizing agents include, but are not limited to carrier proteins, such as albumin, gelatin, metals or inorganic salts. Pharmaceutically acceptable excipient that may be present can further be found in the relevant literature, for example in the Handbook of Pharmaceutical Additives: An International Guide to More Than 6000 Products by Trade Name, Chemical, Function, and Manufacturer; Michael and Irene Ash (Eds.); Gower Publishing Ltd.; Aldershot, Hampshire, England, 1995.

“Compressed fluid” as used herein refers to a fluid of appreciable density (e.g., >0.2 g/cc) that is a gas at standard temperature and pressure. “Supercritical fluid”, “near-critical fluid”, “near-supercritical fluid”, “critical fluid”, “densified fluid” or “densified gas” as used herein refers to a compressed fluid under conditions wherein the temperature is at least 80% of the critical temperature of the fluid and the pressure is at least 50% of the critical pressure of the fluid.

Examples of substances that demonstrate supercritical or near critical behavior suitable for the present invention include, but are not limited to carbon dioxide, isobutylene, ammonia, water, methanol, ethanol, ethane, propane, butane, pentane, dimethyl ether, xenon, sulfur hexafluoride, halogenated and partially halogenated materials such as chlorofluorocarbons, hydrochlorofluorocarbons, hydrofluorocarbons, perfluorocarbons (such as perfluoromethane and perfuoropropane, chloroform, trichloro-fluoromethane, dichloro-difluoromethane, dichloro-tetrafluoroethane) and mixtures thereof.

“Sintering” as used herein refers to the process by which parts of the matrix or the entire polymer matrix becomes continuous (e.g., formation of a continuous polymer film). As discussed below, the sintering process is controlled to produce a fully conformal continuous matrix (complete sintering) or to produce regions or domains of continuous coating while producing voids (discontinuities) in the matrix. As well, the sintering process is controlled such that some phase separation is obtained between polymer different polymers (e.g., polymers A and B) and/or to produce phase separation between discrete polymer particles. Through the sintering process, the adhesions properties of the coating are improved to reduce flaking of detachment of the coating from the substrate during manipulation in use. As described below, in some embodiments, the sintering process is controlled to provide incomplete sintering of the polymer matrix. In embodiments involving incomplete sintering, a polymer matrix is formed with continuous domains, and voids, gaps, cavities, pores, channels or, interstices that provide space for sequestering a therapeutic agent which is released under controlled conditions. Depending on the nature of the polymer, the size of polymer particles and/or other polymer properties, a compressed gas, a densified gas, a near critical fluid or a super-critical fluid may be employed. In one example, carbon dioxide is used to treat a substrate that has been coated with a polymer and a drug, using dry powder and RESS electrostatic coating processes. In another example, isobutylene is employed in the sintering process. In other examples a mixture of carbon dioxide and isobutylene is employed.

When an amorphous material is heated to a temperature above its glass transition temperature, or when a crystalline material is heated to a temperature above a phase transition temperature, the molecules comprising the material are more mobile, which in tum means that they are more active and thus more prone to reactions such as oxidation. However, when an amorphous material is maintained at a temperature below its glass transition temperature, its molecules are substantially immobilized and thus less prone to reactions. Likewise, when a crystalline material is maintained at a temperature below its phase transition temperature, its molecules are substantially immobilized and thus less prone to reactions. Accordingly, processing drug components at mild conditions, such as the deposition and sintering conditions described herein, minimizes cross-reactions and degradation of the drug component. One type of reaction that is minimized by the processes of the invention relates to the ability to avoid conventional solvents which in turn minimizes autoxidation of drug, whether in amorphous, semi-crystalline, or crystalline form, by reducing exposure thereof to free radicals, residual solvents and autoxidation initiators.

“Rapid Expansion of Supercritical Solutions” or “RESS” as used herein involves the dissolution of a polymer into a compressed fluid, typically a supercritical fluid, followed by rapid expansion into a chamber at lower pressure, typically near atmospheric conditions. The rapid expansion of the supercritical fluid solution through a small opening, with its accompanying decrease in density, reduces the dissolution capacity of the fluid and results in the nucleation and growth of polymer particles. The atmosphere of the chamber is maintained in an electrically neutral state by maintaining an isolating “cloud” of gas in the chamber. Carbon dioxide or other appropriate gas is employed to prevent electrical charge is transferred from the substrate to the surrounding environment.

“Bulk properties” properties of a coating including a pharmaceutical or a biological agent that can be enhanced through the methods of the invention include for example: adhesion, smoothness, conformality, thickness, and compositional mixing.

“Electrostatically charged” or “electrical potential” or “electrostatic capture” as used herein refers to the collection of the spray-produced particles upon a substrate that has a different electrostatic potential than the sprayed particles. Thus, the substrate is at an attractive electronic potential with respect to the particles exiting, which results in the capture of the particles upon the substrate. i.e. the substrate and particles are oppositely charged, and the particles transport through the fluid medium of the capture vessel onto the surface of the substrate is enhanced via electrostatic attraction. This may be achieved by charging the particles and grounding the substrate or conversely charging the substrate and grounding the particles, or by some other process, which would be easily envisaged by one of skill in the art of electrostatic capture.

The present invention provides several advantages which overcome or attenuate the limitations of current technology for bioabsorbable stents.

One embodiment provides a coated coronary stent, comprising: a stent framework; heparin molecules attached to the stent framework; and a rapamycin-polymer coating wherein at least part of rapamycin is in crystalline form. In one embodiment, the rapamycin-polymer coating comprises one or more resorbable polymers.

In another embodiment the rapamycin-polymer coating has substantially uniform thickness and rapamycin in the coating is substantially uniformly dispersed within the rapamycin-polymer coating.

In another embodiment, the one or more resorbable polymers are selected from PLGA (poly(lactide-co-glycolide); DLPLA-poly(dl-lactide); LPLA-poly(l-lactide); PGA-polyglycolide; PDO-poly(dioxanone); PGA-TMC-poly(glycolide-co-trimethylene carbonate); PGA-LPLA-poly(l-lactide-co-glycolide); PGA-DLPLA-poly(dl-lactide-co-glycolide); LPLA-DLPLA-poly(l-lactide-co-dl-lactide); PDO-PGA-TMC-poly(glycolide-co-trimethylene carbonate-co-dioxanone) and combinations thereof.

In another embodiment the stent provides an elution profile wherein about 10% to about 50% of rapamycin is eluted at week 1 after the composite is implanted in a subject under physiological conditions, about 25% to about 75% of rapamycin is eluted at week 2 and about 50% to about 100% of rapamycin is eluted at week 6.

A further embodiment provides a coated coronary stent, comprising: a stent framework; heparin molecules attached to the stent framework by an aminated silane; and a rapamycin-polymer coating wherein at least part of rapamycin is in crystalline form and wherein the polymer is bioabsorbable.

Still another embodiment provides a coated coronary stent, comprising: a stent framework having a heparin coating disposed thereon; and a macrolide immunosuppressive (limus) drug-polymer coating wherein at least part of the drug is in crystalline form.

In another embodiment the macrolide immunosuppressive drug comprises one or more of rapamycin, 40-O-(2-Hydroxyethyl)rapamycin (everolimus), 40-O-Benzyl-rapamycin, 40-O-(4′-Hydroxymethyl)benzyl-rapamycin, 40-O-[4′-(1,2-Dihydroxyethyl)]benzyl-rapamycin, 40-O-Allyl-rapamycin, 40-O-[3′-(2,2-Dimethyl-1,3-dioxolan-4(S)-yl)-prop-2′-en-1′-yl]-rapamycin, (2′:E,4′S)-40-O-(4′,5′-Dihydroxypent-2′-en-1′-yl)-rapamycin 40-O-(2-Hydroxy)ethoxycar-bonylmethyl-rapamycin, 40-O-(3-Hydroxy)propyl-rapamycin 40-O-(6-Hydroxy)hexyl-rapamycin 40-O-[2-(2-Hydroxy)ethoxy]ethyl-rapamycin 40-O-[(3S)-2,2-Dimethyldioxolan-3-yl]methyl-rapamycin, 40-O-[(2S)-2,3-Dihydroxyprop-1-yl]-rapamycin, 40-O-(2-Acetoxy)ethyl-rapamycin 40-O-(2-Nicotinoyloxy)ethyl-rapamycin, 40-O-[2-(N-Morpholino)acetoxy]ethyl-rapamycin 40-O-(2-N-Imidazolylacetoxy)ethyl-rapamycin, 40-O-[2-(N-Methyl-N′-piperazinyl)acetoxy]ethyl-rapamycin, 39-0-Desmethyl-39,40-O,0-ethylene-rapamycin, (26R)-26-Dihydro-40-O-(2-hydroxy)ethyl-rapamycin, 28-0-Methyl-rapamycin, 40-O-(2-Aminoethyl)-rapamycin, 40-O-(2-Acetaminoethyl)-rapamycin 40-O-(2-Nicotinamidoethyl)-rapamycin, 40-O-(2-(N-Methyl-imidazo-2′-ylcarbethoxamido)ethyl)-rapamycin, 40-O-(2-Ethoxycarbonylaminoethyl)-rapamycin, 40-O-(2-Tolylsulfonamidoethyl)-rapamycin, 40-O-[2-(4′,5′-Dicarboethoxy-1′,2′,3′-triazol-1′-yl)-ethyl]-rapamycin, 42-Epi-(tetrazolyl)rapamycin (tacrolimus), and 42-[3-hydroxy-2-(hydroxymethyl)-2-methylpropanoate]rapamycin (temsirolimus).

In another embodiment the macrolide immunosuppressive drug is at least 50% crystalline.

Another embodiment provides a method for preparing a coated coronary stent comprising the following steps: forming a silane layer on a stainless or cobalt -chromium stent framework; covalently attaching heparin molecules to the silane layer; forming a macrolide immunosuppressive (limus) drug-polymer coating on the stent framework wherein at least part of the drug is in crystalline form.

In another embodiment the macrolide is deposited in dry powder form.

In another embodiment the bioabsorbable polymer is deposited in dry powder form.

In another embodiment the polymer is deposited by an e-SEDS process.

In another embodiment the polymer is deposited by an e-RESS process.

Another embodiment provides a method further comprising sintering said coating under conditions that do not substantially modify the morphology of said macrolide.

Yet another embodiment provides a coated coronary stent, comprising: a stent framework; heparin molecules attached to the stent framework; a first layer of bioabsorbable polymer; and a rapamycin-polymer coating wherein comprising rapamycin and a second bioabsorbable polymer wherein at least part of rapamycin is in crystalline form and wherein the first polymer is a slow absorbing polymer and the second polymer is a fast absorbing polymer.

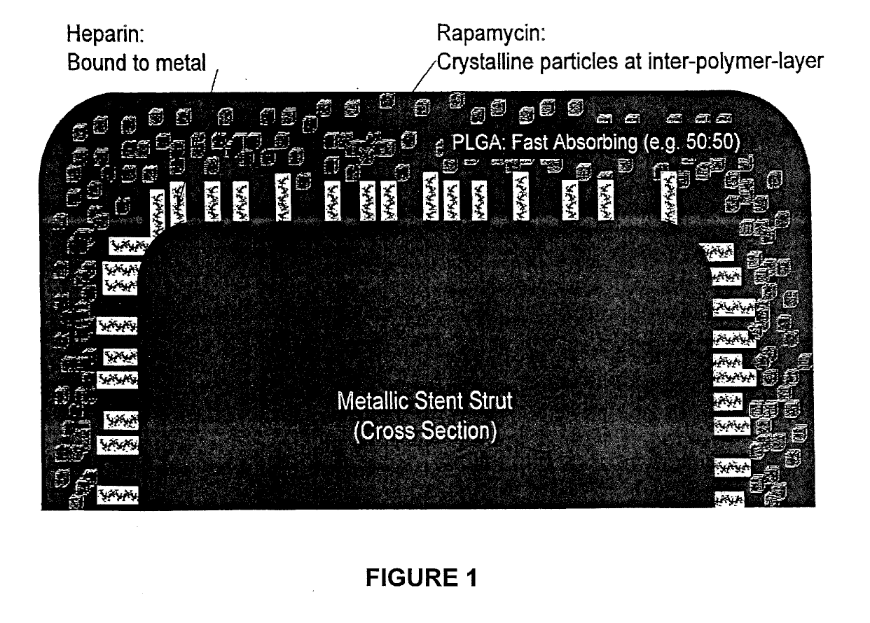

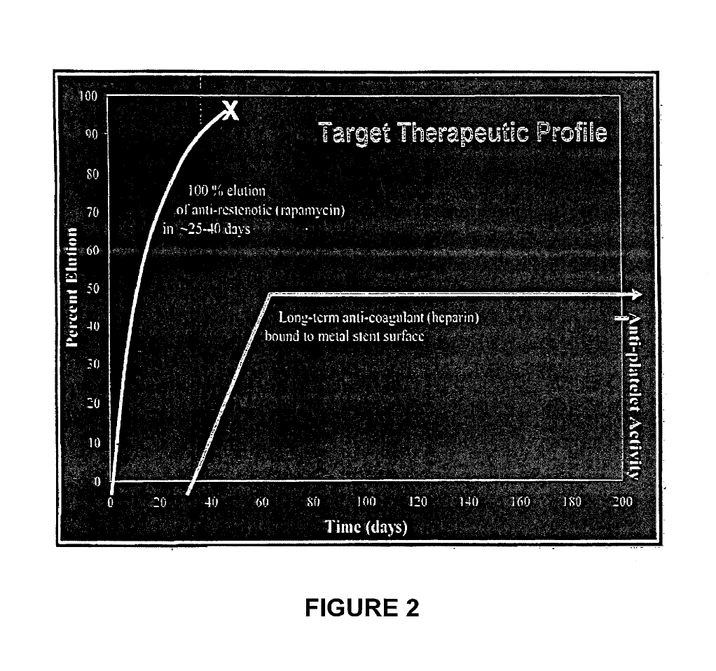

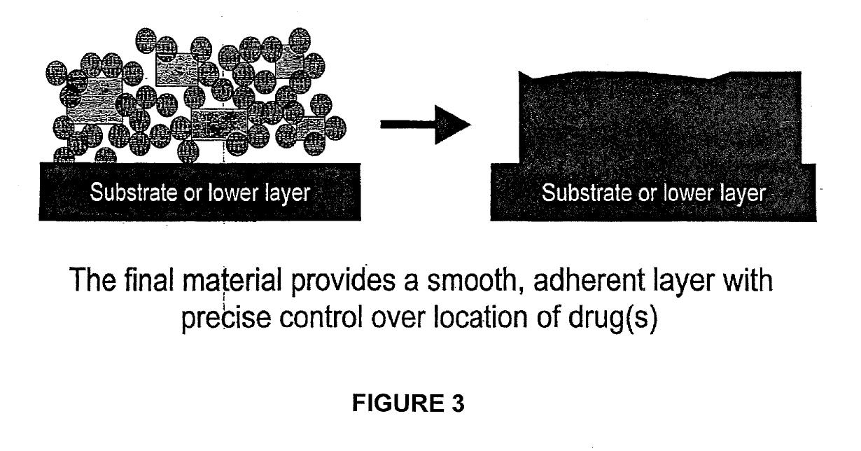

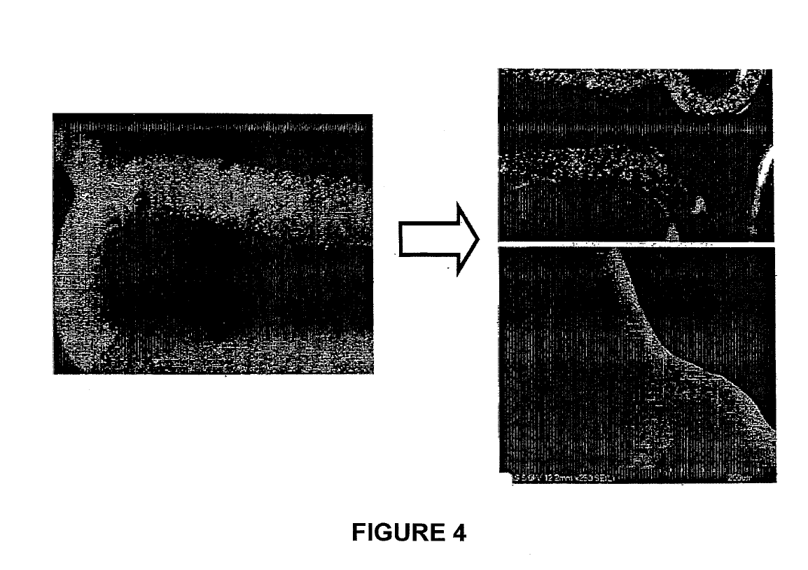

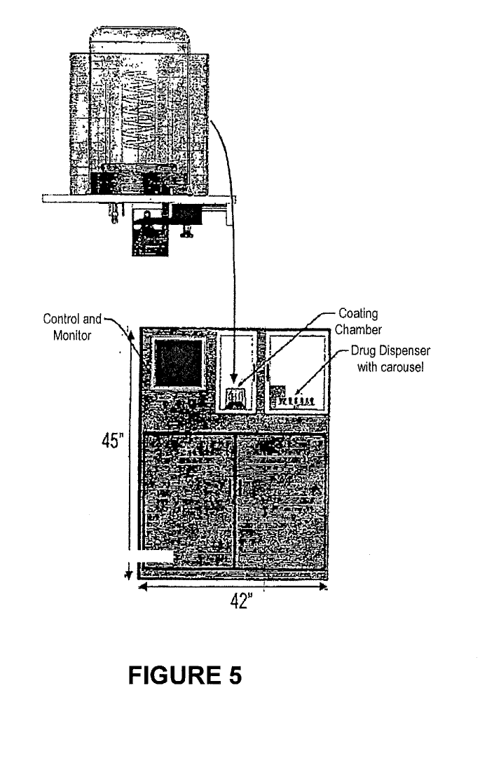

Illustrative embodiments of the present invention are provided in appended FIGS. 1-5.

The foregoing is illustrative of the present invention, and is not to be construed as limiting thereof. While embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A method for preparing a coated coronary stent comprising the following steps:
forming a silane layer on a stainless or cobalt-chromium stent framework;
covalently attaching heparin molecules to the silane layer; and
forming a macrolide immunosuppressive (limus) drug-polymer coating on the stent framework wherein at least part of the drug is in crystalline form and present on an exterior surface of the stent.

2. The method of claim 1 wherein the macrolide is deposited in dry powder form.

3. The method of claim 1 wherein the bioabsorbable polymer is deposited in dry powder form.

4. The method of claim 1 wherein the polymer is deposited by an e-SEDS process.

5. The method of claim 1 wherein the polymer is deposited by an e-RESS process.

6. The method of claim 1 further comprising sintering said coating under conditions that do not substantially modify the morphology of said macrolide.

7. The method of claim 1, wherein the macrolide immunosuppressive drug comprises one or more of rapamycin, 40-0-(2-Hydroxyethyl)rapamycin (everolimus), 40-0-Benzyl rapamycin, 40-0-(4′-Hydroxymethyl)benzyl-rapamycin, 40-0-[4′-(1,2-Dihydroxyethyl)]benzyl-rapamycin, 40-0-Allyl-rapamycin, 40-0-[3′-(2,2-Dimethyl-1,3-dioxolan-4(S)-yl)-prop-2′-en-1′-yl]-rapamycin, (2′:E,4′S)-40-0-(4′,5′-Dihydroxypent-2′ en-1′-yl)-rapamycin 40-0-(2-Hydroxy)ethoxycarbonylmethyl-rapamycin, 40-0-(3-Hydroxy)propyl-rapamycin 40-0-(6-Hydroxy)hexyl-rapamycin 40-0-[2-(2-Hydroxy)ethoxy]ethyl-rapamycin 40-0-[(3S)-2,2-Dimethyldioxolan-3-yl]methyl rapamycin, 40-0-[(2S)-2,3-Dihydroxyprop-1-yl]-rapamycin, 40-0-(2-Acetoxy)ethyl-rapamycin 40-0-(2-Nicotinoyloxy)ethyl-rapamycin, 40-0-[2(N-Morpholino) acetoxy]ethyl-rapamycin 40-0-(2-N-Imidazolylacetoxy)ethyl-rapamycin, 40-0-[2-(N-Methyl-N′-piperazinyl)acetoxy]ethyl-rapamycin, 39-0-Desmethyl-39,40-0,0-ethylene-rapamycin, (26R)-26-Dihydro-40-0-(2-hydroxy)ethyl-rapamycin, 28-0-Methyl-rapamycin, 40-0-(2-Aminoethyl)-rapamycin, 40-0-(2-Acetaminoethyl)-rapamycin 40-0-(2-Nicotinamidoethyl)-rapamycin, 40-0-(2-(N-Methyl-imidazo-2′-ylcarbethoxamido)ethyl)-rapamycin, 40-0-(2-Ethoxycarbonylaminoethyl)-rapamycin, 40-0-(2-Tolylsulfonamidoethyl)-rapamycin, 40-0-[2-(4′,5′-Dicarboethoxy-1′,2′,3′-triazol-1′-yl)-ethyl]-rapamycin, 42-Epi-(tetrazolyl)rapamycin (tacrolimus), and 42-[3-hydroxy-2-(hydroxymethyl)-2-methylpropanoate]rapamycin (temsirolimus).

8. The method of claim 1 wherein one or more resorbable polymers are selected from PLGA (poly(lactide-co-glycolide); DLPLA-poly(dl-lactide); LPLA-poly(l-lactide); PGA-polyglycolide; PDQ-poly(dioxanone); PGA-TMC-poly(glycolide-co trimethylene carbonate); PGA-LPLA-poly(l-lactide-co-glycolide); PGA-DLPLA poly(dl-lactide-co-glycolide); LPLA-DLPLA-poly(l-lactide-co-dl-lactide); PDO-PGA-TMC-poly(glycolide-co-trimethylene carbonate-co-dioxanone).

9. A method for preparing a coated implantable device comprising the steps of:
forming a silane layer on a metallic substrate;
covalently attaching heparin molecules to the silane layer; and
forming a coating including a drug and a polymer over the heparin molecules, wherein at least part of the drug is in crystalline form and present on an exterior surface of the device.

10. The method of claim 9 wherein the drug is deposited in dry powder form.

11. The method of claim 9 wherein the polymer is deposited in dry powder form.

12. The method of claim 9 wherein the polymer is deposited by an e-SEDS process.

13. The method of claim 9 wherein the polymer is deposited by an e-RESS process.

14. The method of claim 9 further comprising sintering the coating under conditions that do not substantially modify the morphology of the drug.

15. The method of claim 9, wherein the drug comprises one or more of rapamycin, 40-0-(2-Hydroxyethyl)rapamycin (everolimus), 40-0-Benzyl rapamycin, 40-0-(4′-Hydroxymethyl)benzyl-rapamycin, 40-0-[4′-(1,2-Dihydroxyethyl)]benzyl-rapamycin, 40-0-Allyl-rapamycin, 40-0-[3′-(2,2-Dimethyl-!,3-dioxolan-4(S)-yl)-prop-2′-en-1′-yl]-rapamycin, (2′:E,4′S)-40-0-(4′,5′-Dihydroxypent-2′ en-1′-yl)-rapamycin 40-0-(2-Hydroxy)ethoxycar-bonylmethyl-rapamycin, 40-0-(3-Hydroxy)propyl-rapamycin 40-0-(6-Hydroxy)hexyl-rapamycin 40-0-[2-(2-Hydroxy)ethoxy]ethyl-rapamycin 40-0-[(3S)-2,2-Dimethyldioxolan-3-yl]methyl rapamycin, 40-0-[(2S)-2,3-Dihydroxyprop-1-yl]-rapamycin, 40-0-(2-Acetoxy)ethyl-rapamycin 40-0-(2-Nicotinoyloxy)ethyl-rapamycin, 40-0-[2-(N-Morpholino) acetoxy]ethyl-rapamycin 40-0-(2-N-Imidazolylacetoxy)ethyl-rapamycin, 40-0-[2-(N-Methyl-N′-piperazinyl)acetoxy]ethyl-rapamycin, 39-0-Desmethyl-39,40-0,0-ethylene-rapamycin, (26R)-26-Dihydro-40-0-(2-hydroxy)ethyl-rapamycin, 28-0-Methyl-rapamycin, 40-0-(2-Aminoethyl)-rapamycin, 40-0-(2-Acetaminoethyl)-rapamycin 40-0-(2-Nicotinamidoethyl)-rapamycin, 40-0-(2-(N-Methyl-imidazo-2′-ylcarbethoxamido)ethyl)-rapamycin, 40-0-(2-Ethoxycarbonylaminoethyl)-rapamycin, 40-0-(2-Tolylsulfonamidoethyl)-rapamycin, 40-0-[2-(4′,5′-Dicarboethoxy-1′,2′,3′-triazol-1′-yl)-ethyl]-rapamycin, 42-Epi-(tetrazolyl)rapamycin (tacrolimus), and 42-[3-hydroxy-2-(hydroxymethyl)-2-methylpropanoate]rapamycin (temsirolimus).

16. The method of claim 9 wherein the polymer is selected from PLGA (poly(lactide-co-glycolide); DLPLA-poly(dl-lactide); LPLA-poly(l-lactide); PGA-polyglycolide;
PDQ-poly(dioxanone); PGA-TMC-poly(glycolide-co trimethylene carbonate); PGA-LPLA-poly(l-lactide-co-glycolide); PGA-DLPLA poly(dl-lactide-co-glycolide); LPLA-DLPLA-poly(l-lactide-co-dl-lactide); PDO-PGA-TMC-poly(glycolide-co-trimethylene carbonate-co-dioxanone).

17. A method for preparing a coated implantable device comprising the steps of:
forming a silane layer on a metallic substrate;
covalently attaching heparin molecules to the silane layer;
depositing a drug in dry powder form over the heparin molecules;
depositing a polymer in dry powder form over the heparin molecules; and
forming a coating including the drug and the polymer over the heparin molecules, wherein at least part of the drug is in crystalline form and present on an exterior surface of the device.

18. The method of claim 17 wherein the forming step includes sintering.

19. The method of claim 18 wherein the sintering step is performed under conditions that do not substantially modify the morphology of the drug.

20. The method of claim 17 wherein the drug is a macrolide immunosuppressive (limus) drug.
